SEQUENCE LISTING (1 sequence)

SEQ ID NO: 1 - DNA, 1132 bp, Artificial Sequence
  CAG fusion promoter
ggccgctctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaa

(19) United States
(12) Patent Application Publication
(10) Pub. No.: US 2010/0255505 A1
(43) Pub. Date: Oct. 7, 2010

(54) GENETICALLY MODIFIED STEM CELLS AND METHODS FOR IDENTIFYING TISSUES DIFFERENTIATED THEREFROM

(76) Inventors: Agota Apáti, Budapest (HU); Zsuzsa Izsvák, Gyongyos (HU); Katalin Német, Budapest (HU); Tamás Orbán, Budapest (HU); Balázs Sarkadi, Budapest (HU)

Correspondence Address: HAHN & VOIGHT PLLC, 1012 14TH STREET, NW, SUITE 620, WASHINGTON, DC, 20005, US

(21) Appl. No.: 12/780,315

(22) Filed: May 14, 2010

Related U.S. Application Data
(63) Continuation-in-part of application No. PCT/IB2008/054238, filed on Oct. 15, 2008.

(30) Foreign Application Priority Data
Oct. 15, 2007 (HU) P0700675

(51) Int. Cl. G01N 33/53; C12N 5/071

(52) U.S. Cl. 435/7.21; 435/325; 435/366; 435/377

(57) ABSTRACT

Genetically modified stem cells and the selection of cells differentiated therefrom are disclosed. Particularly, the herein disclosed invention relates to stem cells or cells differentiated therefrom containing a copy of a stably inheritable expression construct that is suitable for the expression of transgenes in stem cells, wherein said construct comprises at least a double-feature constitutive promoter being operable both in stem cells and in differentiated tissues, the expression level thereof being subject to a tissue or cell type specific regulation in differentiated cells, and, optionally, under the control of said promoter, a transgene, wherein said transgene is expressed in the stem cell. Furthermore methods are disclosed to produce such stem cells, as well as specific uses of said stem cells in assay methods and in human therapy and in veterinary practice.

This is a continuation-in-part of International Application PCT/IB2008/054238, filed Oct. 15, 2008, which claims priority to Hungarian Application No. P0700675, filed Oct. 15, 2007, the entire disclosures of which are hereby incorporated by reference herein.

### FIELD OF THE INVENTION

This invention relates generally to genetically modified stem cells and to the selection of cells differentiated therefrom. Particularly, the herein disclosed invention relates to stem cells or cells differentiated therefrom containing a copy of a stably inheritable expression construct that is suitable for the expression of transgenes in stem cells, wherein said construct comprises at least a double-feature constitutive promoter being operable both in stem cells and in differentiated tissues, the expression level thereof being subject to a tissue or cell type specific regulation in differentiated cells, and, optionally, under the control of said promoter, a transgene, wherein said transgene is expressed in the stem cell. Furthermore methods are disclosed to produce such stem cells, as well as specific uses of said stem cells in assay methods and in human therapy and in veterinary practice.

### BACKGROUND OF THE INVENTION

The application of stem cells provides new hopes in the clinical treatment of a number of diseases and, at the same time, they are excellent models for tissue development and physiological cell differentiation. The so called “regenerative medicine” makes use of cells that can grow and differentiate to replace a damaged tissue. Also, efficient and stable gene delivery into stem cells should form the basis of successful gene therapy applications.

Human embryonic stem (HuES) cells represent a great potential for gene therapy purposes. Nevertheless, one of the major difficulties of HuES-based applications is to achieve efficient, stable gene delivery and to conduct a directed tissue differentiation and separation. This would undoubtedly provide useful tools in medical research and practice, providing large quantities of reproducible model systems for various medically relevant diseases.

Current methods for directed tissue differentiation often apply endogenous morphogenetic proteins or invasive chemicals on HuES cells to obtain the tissue(s) of interest (Lev, S et al., Ann N Y Acad Sci 2005, 1047:50; Karner, E et al., Stem Cells Dev 2007, 16:39; Chen, K et al., J Cell Biochem 2008, 104:119). However, the use of such artificial cocktail of drugs is far from natural differentiation and, among other problems, could probably induce undesired gene expression profiles. In addition, it is difficult to prove that tissues derived from such invasive attempts reliably represent naturally occurring cell types (Tomescot, A et al., Stem Cells 2007, 25:2200). Currently, the most widely applied methods for gene delivery into various stem/progenitor cells are based on the use of viral vector constructs. By now, there are numerous efficient retrovirus-lentivirus- or other virus-based methods which allow stable genomic incorporation of the foreign DNAs with high gene product expression levels (see e.g. WO 06126972). However, virus-based gene therapy technologies also have serious drawbacks, including safety concerns of virus production, and the preferential insertion of virus into active genes, which might cause uncontrolled proliferation of the gene-modified stem cells (Schroder, A R et al., Cell 2002, 110:521; VandenDriessche, T et al., Curr Gene Ther 2003, 3:501).

A common way of overcoming the above problems is to use a combination of tissue-specific promoters and certain drug resistance genes as selection markers. This method can provide the solution for obtaining the desired cell types since it is based on “spontaneous” differentiation of HuES cells (at least without the induction of invasive chemicals, mimicking more natural conditions, see Passier, R et al., Stem Cells 2005, 23:772) and selecting for the differentiated cell types in a latter phase, eg. selecting by certain antibiotics (Kolossov, E et al., J Exp Med 2006, 203:2315; Huber, I et al., FASEB J 2007, 21:2551). In principle, this could result in the enrichment of desired tissues in large quantities and most likely providing less interference with the gene expression profiles of the tissues of interest. Although better than using artificial chemicals, it requires viral or non-viral gene delivery into HuES cells which itself can be technically challenging, and antibiotic selection could also induce a certain way of differentiation, again asking for the need to prove that the obtained tissues represent naturally occurring cell types (Duan, Y et al., Stem Cells 2007, 25:3058).

The usage of tissue specific promoters generates another technical problem. When using a promoter with high tissue specificity (which is certainly required by this application), the transgene expression will not be detectable in undifferentiated HuES cells (Gallo, P et al., Gene Ther 2008, 15:161). Especially for non-viral applications, where the efficiency of delivery is generally quite low, this represents a serious disadvantage. To avoid this, one can use another constitutive promoter to drive the expression of an additional selection marker gene to first select for transgene delivery and expression, then after spontaneous differentiation, for the forming of specific tissues. Apart from being cumbersome and time-consuming, this also creates another technically challenging point since it either means the co-delivery of different transgenes (eg. co-transfection with two plasmids) or a delivery of a larger genetic cargo, often resulting in lower delivery efficiency.

The solution for the above stated problems could be a promoter that is operable in stem cells and at the same time has tissue specific regulation in differentiated cells. Such a promoter would have many advantages, by using such a promoter there would be no need for using viruses and the selection of differentiated cells could be done with the same construct. An ideal solution would be a constitutive promoter that is operable both in stem cells and in differentiated tissues, the expression level thereof being subject to a tissue or cell type specific regulation in differentiated cells. Such a promoter would enable the expression of an introduced transgene that could be expressed in the stem cell or the cell differentiated therefrom.

Therefore the object of the present invention was to develop an application of a non-viral methodology for efficient and stable gene delivery into stem cells, preferably into HuES cells.

In the detailed description below, we shall demonstrate several examples concerning stem cells, the cells differentiated from said stem cells and the methods according to the present invention, etc. However it is obvious for a person skilled in the art that herein only certain specific embodiments of the present invention are described to assist the person skilled in the pertinent art. Clearly, we have no intention to limit the scope of the present invention with the described examples, they are only assisting in the use of the present invention.

The term “double-feature” constitutive promoter within the context of this invention refers to a promoter that enables constitutive expression in stem cells, but at the same time drives expression differently in different types of differentiated cells or tissues.

### SUMMARY OF THE INVENTION

The technical solution according to the present invention is based on the surprising and unexpected finding, that certain promoters, like the CAG promoter (Niwa, H et al., Gene 1991, 108:193) are capable of driving a constitutive expression in stem cells while at the same time the expression driven by said promoters is tissue or cell type specific in differentiated cells, therefore the said promoters are double-feature constitutive promoters. Particularly the expression of the CAG promoter was found to be extremely strong in cardiomyocytes. This unexpected behavior in itself enables the selection of differentiated cardiomyocytes.

The term “CAG promoter” refers to a promoter that is a fusion promoter and generally contains the Cytomegalovirus (CMV) Immediate Early Enhacer, two parts of the chicken beta-actin promoter and part of the rabbit beta1-globin promoter (see FIG. 1). More specifically, in the 1132 by long promoter constructs, nucleotide region 15-380 corresponds to the CMV Immediate Early Enhancer, nucleotide regions 381-861 and 861-1014 correspond to the two parts of the chicken beta-actin promoter sequences, and nucleotide region 1023-1126 represents the rabbit beta1-globin promoter sequence. Other short sequences on the construct represent linker sequences (Niwa, H et al., Gene 1991, 108:193). According to a preferred embodiment of the present invention the CAG promoter's sequence is the sequence given in SEQ ID NO:1 or its functional double-feature homologue.

### DETAILED DESCRIPTION OF THE INVENTION

The present invention relates to stem cells or cells differentiated therefrom containing a copy of a stably inheritable expression construct that is suitable for the expression of transgenes in stem cells, said construct comprising:
at least a double-feature promoter being operable both in stem cells and in differentiated tissues, wherein said promoter is
   constitutive while
   the expression level thereof being subject to a tissue or cell type specific regulation in differentiated cells, and, optionally,
under the control of said promoter, a transgene, wherein said transgene is expressed in the stem cell.

According to a preferred embodiment, the above expression construct further comprises viral transduction or transposon derived sequences.

According to a further preferred embodiment, said promoter is selected from a group consisting of a fusion promoter, preferably a double-feature CAG promoter; a cardiac actin promoter and a human albumin promoter. The use of the double-feature CAG promoter is preferred which according to a preferred embodiment contains the Cytomegalovirus (CMV) Immediate Early Enhancer, two parts of the chicken beta-actin promoter and part of the rabbit beta1-globin promoter. According to a still further preferred embodiment of the present invention the nucleotide sequence of the double-feature CAG promoter is the sequence given in SEQ ID NO:1 or its functional double-feature homologue.

According to a still further preferred embodiment of the invention the above expression construct further comprises a pair of sequences allowing site-specific recombination upstream of the promoter and downstream of said transgene and, preferably, the stem cell comprises a further construct enabling the expression of
a site specific recombinase and
a further transgene controlled by a promoter flanked by a pair of sequences allowing site-specific recombination.

Preferably said pair of sequences allowing site-specific recombination are the Lox sites of the Cre recombinase or the FRT sites of the FLP recombinase, and the site-specific recombinase is the Cre recombinase or the FLP recombinase, respectively.

According to a preferred embodiment of the present invention said transgene is a reporter gene and a reporter protein encoded by said reporter gene is expressed in said stem cell, wherein preferably said reporter protein gives rise to a detectable signal the intensity of which correlates with the activity of said promoter or with cellular conditions or said reporter protein gives specific resistance to a chemical agent which resistance correlates with the activity of said promoter or with cellular conditions. A person skilled in the art would recognize that the specific function of said transgene is not to be construed as limiting to the present invention as long as it can be expressed in stem cells. Therefore said transgene can be a construct for siRNA expression and a connected reporter gene, and the siRNA and the reporter protein encoded by said reporter gene are expressed in said stem cell or in its differentiated derivative, wherein preferably said reporter protein gives a detectable signal the intensity of which correlates with the activity of said promoter or with cellular conditions. However, preferably, said reporter protein is a fluorescent protein selected from a group comprising Green Fluorescent Protein (GFP), Ca-sensitive GFP (GCaMP2), Red Fluorescent Protein (RFP), Yellow Fluorescent Protein (YFP), Cyan Fluorescent Protein (CFP), DS-redMS T Fluorescent Protein; Luciferase; β-galactosidase; a sensor fluorescence protein for reporting cellular ion concentrations or membrane potential or a protein providing resistance to chemical selecting agents, advantageously to puromycin, neomycin or other cytotoxic agent.

The stem cell of the invention can be an animal or a human stem cell. Furthermore said stem cell can be animal or human embryonic stem cell. If human embryonic stem cell is used, then it was produced without causing any harm to a human embryo or preferably said stem cell is not derived from any cell that was produced by causing any harm to a human embryo. Preferably the stem cell of the invention is a human embryonic stem cell (HUES) or a human derived induced pluripotent stem (iPS) cell.

The invention further concerns a cell differentiated from the stem cell of the invention, wherein said stem cell comprises a stably inherited or stably inserted expression construct that is suitable for expression of a transgene in stem cells, said construct comprising at least the above defined constitutive promoter which is operable in stem cells, wherein the expression level of said promoter is subject to a tissue or cell type specific regulation, and, optionally, a transgene under the control of said promoter.

The invention also relates to a method for producing a stem cell or a differentiated stem cell according to the invention, comprising the following steps:

i) providing a construct that comprises
at least a constitutive promoter being operable both in stem cells and in differentiated tissues, and having an expression level being subject to a tissue or cell type specific regulation in differentiated cells, and, optionally,
under the control of said promoter, a transgene, wherein said transgene is expressed in said stem cell
   ii) introducing the construct as defined in step i) into a stem cell and, optionally,
   iii) differentiating said stem cell.

According to a preferred embodiment said introduction of said construct in step ii) is done by viral transduction or by using a transposon-transposase system. The used transposon-transposase system could be the well known Sleeping Beauty or the newly discovered Frog Prince transposon-transposase system (for the description of the latter see e.g. EP1507865). Said promoter, according to a preferred embodiment, is selected from a group comprising a fusion promoter, preferably a double-feature CAG promoter; cardiac actin promoter; and human albumin promoter. Preferably a double-feature CAG promoter is used in the present invention and the double-feature CAG promoter contains the Cytomegalovirus (CMV) Immediate Early Enhancer, two parts of the chicken beta-actin promoter and part of the rabbit beta1-globin promoter.

According to a preferred embodiment the above-mentioned expression construct further comprises a pair of sites allowing site-specific recombination upstream of said promoter and downstream of said transgene and, preferably, said stem cell comprises a further vector comprising

i) a pair of sites allowing site-specific recombination and
ii) a further transgene under the control of a promoter, flanked by sites allowing site-specific recombination. According to the invention the further transgene can be any gene that is operable in stem cells or in cells differentiated therefrom, while it is an object of the invention to provide a stem cell into which any transgenes can be easily incorporated.

In a still further preferred embodiment of the invention said expression construct further comprises a pair of sites allowing site-specific recombination upstream of said promoter and downstream of said transgene and, preferably, said stem cell comprises a further construct comprising

i) a pair of sites allowing site-specific recombination and
ii) a construct allowing siRNA expression and connected reporter gene expression flanked by sites allowing site-specific recombination.

According to a still further preferred embodiment of the invention said transgene or further transgene is a reporter gene and a reporter protein encoded by said reporter gene is expressed in said stem cell or differentiated cell, wherein preferably said reporter protein gives a detectable signal the intensity of which correlates with the activity of the promoter. Preferably said reporter protein is a fluorescent protein selected from a group comprising Green Fluorescent Protein (GFP), Ca-sensitive GFP (GCaMP2), Red Fluorescent Protein (RFP), Yellow Fluorescent Protein (YFP), Cyan Fluorescent Protein (CFP), DS-redMST Fluorescent Protein; Luciferase; β-galactosidase; a sensor fluorescence protein for reporting cellular ion concentrations or membrane potential or a protein providing resistance to chemical selecting agents, advantageously to puromycin or neomycin.

The invention further concerns the use of stem cells according to the invention for testing the effect of a test compound on stem cell differentiation.

The invention further relates to a method of identifying or profiling a modulator of cell differentiation comprising:

i) contacting a test sample comprising stem cells according to the invention with a test compound, including drugs, hormones, artificial or natural compounds, and
ii) optionally allowing said stem cells to differentiate;
iii) determining the effect of said test compound on the amount of said reporter gene product or activity in the differentiated cells as compared to a control sample.

The invention still further relates to a method for monitoring stem cell differentiation comprising:

i) providing a stem cell of the present invention,
ii) monitoring the expression level of said reporter gene or the amount or activity of said reporter gene product, and
iii) drawing conclusions regarding the direction of the differentiation of said stem cell based on the expression level of said reporter gene in any of the cells differentiated from said stem cell. According to a preferred embodiment said monitoring is done by measuring the intensity of the signal emitted by the product of said reporter gene.

The invention further concerns a reagent kit comprising:
a stem cell of the present invention, and
one or more test reagents to implement any of the above said methods.

The above method is especially suited to test drugs affecting cardiomyocyte differentiation, as any effect that influences this pathway can be monitored and quantified by exploiting the unique feature of the CAG promoter. Also, the effect of drugs on any other promoter during the above mentioned differentiation pathway may also be examined since cardiomyocytes or their progenitor cells can be studied selectively. In addition, the CAG promoter driven gene expression-based enrichment provides opportunities for high-throughput pharmacological testing of differentiated cardiomyocytes.

The stem cell or a cell differentiated therefrom according to the invention or a stem cell produced according to a method of the invention can be used in the field of human or veterinary therapy and drug testing. The stem cell or a cell differentiated therefrom has a great advantage compared to other stem cells, namely it can be selected without disturbing said stem cell or a cell differentiated therefrom with compounds (e.g. antibiotics or other selecting agents) normally used for the selection of cells. A further advantage in this respect is that the stem cells or the cells differentiated therefrom can be produced without using viruses. This is advantageous since the use of viruses bears an inherent health hazard. The CAG promoter's surprising double-feature behavior, namely it is constitutive in stem cells, but at the same time its expression is extremely strong in cardiomyocytes is a very unique property. Other constitutive promoters (e.g. EF1α, PGK and CMV) were also tested in this respect but these promoters did not show the above double-feature property. Furthermore this behavior is independent of the transgene sequence, as amaxaGFP and EGFP gave the same results as the canonical GFP. Besides, this behavior is independent of the gene delivery method as transposon-based and lentiviral-based methods gave the same results. The exact integration site of the promoter also does not affect this surprising behavior. Moreover this behavior is independent of the transgene copy number.

### BRIEF DESCRIPTION OF THE DRAWINGS

FIG. 1 shows the general structure of the CAG promoter and its nucleotide sequence.

On FIG. 2 transposons and integration sites are depicted. (A) Structure of the used transposons and SB transposase expressing plasmids. The transposon constructs contained either the CAG or the CMV promoters whereas the transposase was only expressed transiently by the CMV promoter. (B) Examples of transposon integration sites in HUES cells determined by splinkerette or inverse PCR; Hs chr.=Homo sapiens chromosome.

FIG. 3 shows human embryonic stem cells stably expressing the GFP transgene. (A) Morphology and GFP expression of human embryonic stem cell (HUES9) clumps. GFP expression detected by a fluorescent light microscope following transfection (left panel), or after subsequent sorting and cloning of the transgene expressing cells (right panel-magnification: 200×). (B) GFP expressing HUES9 clones on mouse embryonic fibroblasts stained for embryonic stem cell markers. The Oct-4 transcription factor is localized in the nucleus, whereas the SSEA-4 protein is localized in the plasma membrane. Merged confocal images, sizes indicated by the scale bars. Blue: Oct-4 (left panel) or SSEA-4 protein (right panel); green: GFP; red: Tric-phalloidin, representing actin filaments; white: DAPI staining for the nucleus in the case of Oct-4 detection. (C) Six days old EBs formed from GFP expressing HUES9 clones in fluorescence microscopy (magnification: 40×).

FIG. 4 shows a typical area of differentiated HUES9 cells with beating cardiomyocytes (white arrows). The pictures represent the prominent difference of GFP expression between the cardiomyocytes and surrounding other tissues (40× magnification). For the CAG promoter, the expression intensity of GFP is always several magnitudes higher in beating cardiomyocytes than in the surrounding other tissue types. This phenomenon is independent of the gene delivery method as transposon-based transgenes (SB-Sleeping Beauty transposon) or lentiviral (LV)-derived transgenes behave similarly. However, this phenomenon cannot be seen when using other constitutive promoters (such as the EF1α promoter). White arrows point to beating cardiomyocytes differentiated from clones of transgenic human embryonic stem cells. The simplified structure of the transgene cassette is indicated on the top of the pictures.

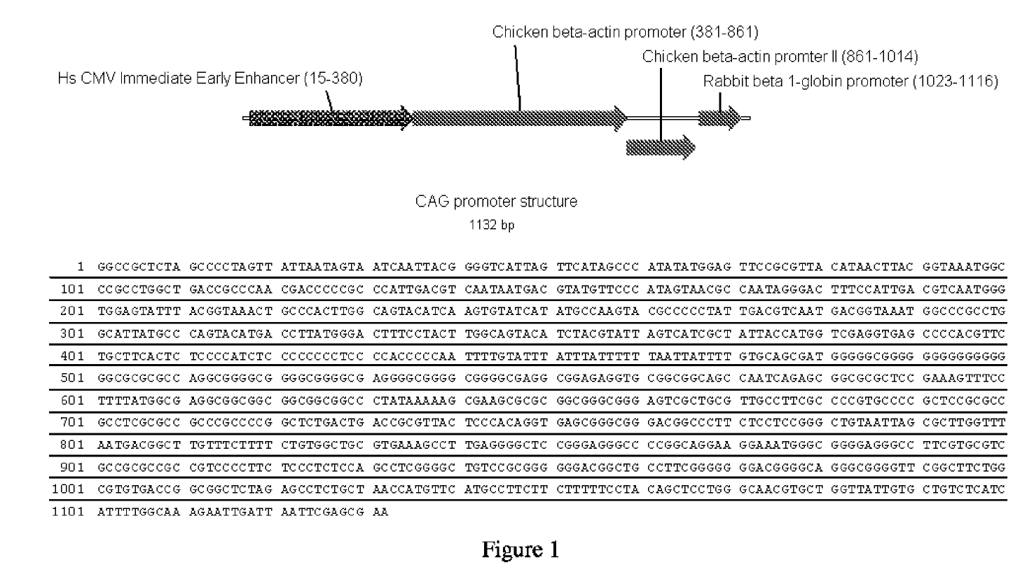

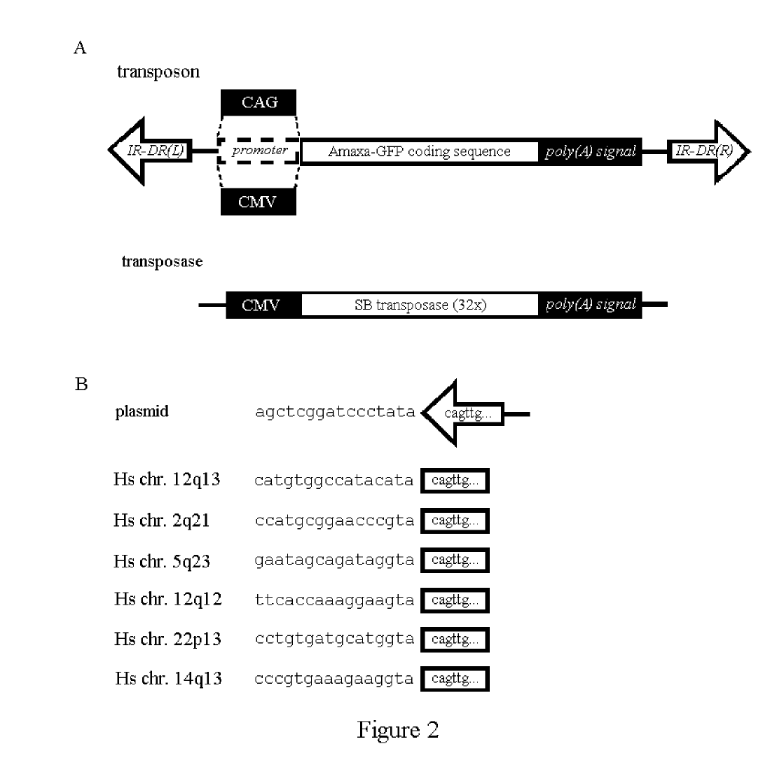

### EXAMPLES

The following examples further illustrate the present invention but should not be construed as in any way limiting its scope.

### Example 1

### Cell Culturing and Differentiation

The human embryonic stem cell line HUES9 was maintained according to the widely accepted culture protocol described in Cowan, Calif. et al., N Engl J Med 2004, 350:1353. Briefly, cells were cultured on mitomycin-C treated mouse embryonic fibroblast feeder cells in complete HUES medium consisting of 15% knockout serum replacement (Gibco, Grand Island, N.Y.), 80% knockout Dulbecco modified Eagle medium (KoDMEM) medium (Invitrogen, Carlsbad, Calif.), 1 mM L-glutamine, 0.1 mM beta-mercaptoethanol, 1% nonessential amino acids, and 4 ng/mL human fibroblast growth factor. HUES cells from passage no. 35 were used for these analyses. The differentiation of the HUES cells were initiated spontaneously. On the day of passage, undifferentiated cells at confluence in 6-well plates were treated with collagenase IV and were transferred to Poly(2 Hydroxyethyl-methacrylate) (Sigma-Aldrich, St. Louis, Mo.) treated Petri dishes to allow EB formation. Cells were kept in differentiation medium consisting of KoDMEM supplemented with 20% non heat-inactivated fetal bovine serum (Invitrogen, Carlsbad, Calif.), 1% nonessential amino acids, 1 mM L-glutamine, and 0.1 mM beta-mercaptoethanol for six days, the medium being changed daily. Under these conditions, the HUES cells generate EBs which are complex, teratoma-like tissue structures with highly variable forms and tissue elements (Lev, S et al., Ann N Y Acad Sci 2005, 1047:50; Thomson, JA et al., Science 1998, 282:1145). These EBs were placed onto gelatin-coated plates, where they attach to the surface and continue a further spontaneous differentiation process by forming recognizable tissue-types. Under these conditions, formation of endothelial, epithelial, and neuronal cells, as well as of fibroblasts and cardiomyocytes could be observed. In this study we applied conditions to provide an enrichment of cardiomyocytes and/or neural precursors (so called rosettes) and differentiated neurons, in addition to the generally appearing fibroblasts in the culture plates (Schulz, T C et al., BMC Neurosci 2003, 4:27). Cell types were identified by morphological signs under phase contrast light microscopy, as well as by immunostaining for various protein markers (see below, Example 5). For puromycin selection (see also Example 2), 0.8 μg/ml puromycin was used for undifferentiated HuES cells.

Human derived induced pluripotent stem (iPS) cells can be maintained and differentiated e.g. as described by Zhang et al. [Circ. Res. 104 e30-e41 (2009)].

### Example 2

### Transposon Constructs and Transfection Procedure

SB transposon plasmids used in this project contained the cDNA of the highly fluorescent marker Amaxa-GFP (www.amaxa.com) (FIG. 2A) or the canonical EGFP between the transposon inverted repeat sequences. Promoter sequences can be obtained from a tissue-specific promoter database, e.g. the TiProD: Tissue-Specific Promoter Database, (http://tiprod.cbi.pku.edu.cn:8080/index.html), including cardiac actin or human albumin promoters. Other chemically inducible promoters, e.g. a metallothionein promoter, or artificial fusion promoters, eg. the CAG promoter can also be utilized. The CAG is a composite promoter that combines the human cytomegalovirus immediate-early enhancer, two parts of the chicken beta-actin promoter, and one part of the rabbit beta1-globin promoter. The CAG promoter is a very strong and ubiquitous promoter. It produces high levels of expression both in vitro and in vivo. The CAG promoter (SEQ ID NO:1) was successfully used to express the enhanced GFP in all tissues of transgenic mice with the exception of erythrocytes and hair. Comparison analyses have shown that the CAG promoter is more efficient than the CMV promoter/enhancer.

By a HindIII and NheI restriction digestion followed by ligation, we cloned the CMV viral promoter, the CAG promoter (FIG. 2A), the human phosphoglycerate kinase (PGK) promoter or the short version of the human elongation factor 1α (EF1α) promoter upstream of the transgene. For the antibiotic selection experiments, the puromycin resistance gene was cloned between the transposon inverted repeat sequences driven by the CAG promoter. For the SB transposase, we used an enhanced version of the protein having 32 times higher transposase activity than the originally reconstructed species [unpublished results]; for transposition control, we applied a DDE mutant form of the protein (Ivics, Z et al., Cell 1997, 91:501). For transfection, the FuGENE® 6 (Roche Applied Science, Switzerland) reagent was used according to the manufacturer's instruction. Before transfection, HUES cells were separated from the mouse feeder cells and placed on gelatin-coated plates. The subsequent day, the cells were co-transfected with transposon and transposase plasmids in a 10:1 ratio to avoid the overproduction inhibition of the transposase (Izsvak, Z et al., Mol Ther 2004, 9:147; Izsvak, Z et al., J Mol Biol 2000, 302:93). Next day, the transfected HUES cells were placed back onto the mouse feeder layer to keep them in an undifferentiated state.

### Example 3

### Detecting Transposon Activity and Determining Transposon Integration Sites

To provide evidence that the transposon construct is capable of transposition, the “excision PCR”, a nested PCR method was applied, as described previously. This method amplifies the “footprint” sequence left on the plasmid after transposition, whereas no PCR product is obtained if no transposition occurs (Ivics, Z et al., Mol Ther 2007, 15:1137). To determine the integration sites of the transgenes in human genomic DNA, splinkerette PCR and inverse PCR methods were applied, essentially as described earlier (Ivics, Z et al., Cell 1997, 91:501; Vigdal, T J et al., J Mol Biol 2002, 323:441). Briefly, for the splinkerette PCR, either a Sau3AI restriction enzyme, or the BfaI/NdeI/AseI enzyme cocktail was used to digest the genomic DNA, followed by a nested PCR using primers described earlier (Ivics, Z et al., Cell 1997, 91:501; Vigdal, T J et al., J Mol Biol 2002, 323:441). For the inverse PCR, the BamHI/Bc1I enzyme cocktail was applied for the digestion of the genomic DNA, followed by a nested PCR using previously described primer pairs (Ivics, Z et al., Cell 1997, 91:501; Vigdal, T J et al., J Mol Biol 2002, 323:441).

### Example 4

### Lentiviral Constructs and Transduction Procedures

For the viral-based gene delivery, the new generation SEW lentiviral vectors were used (Schambach, A et al., Mol Ther 2007, 15:1167). The transgenes used for the experiments were exactly the same as used for the transposon constructs (see Example 2 above), each case removing the transcription unit from the transposon vector by restriction digestion and inserting it into the viral vector by blunt end ligation. Virus titers and transduction procedures were made and performed essentially as described previously (Ujhelly, 0 et al., Hum Gene Ther 2003, 14:403).

### Example 5

### Flow Cytometry

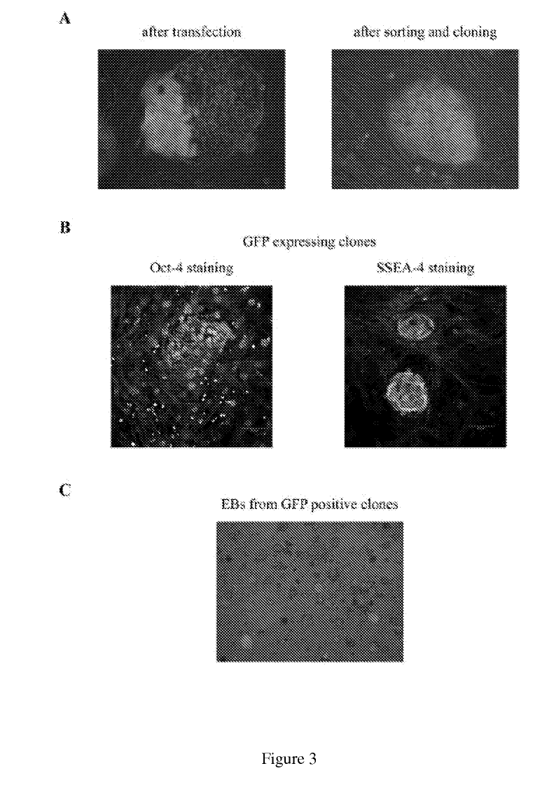

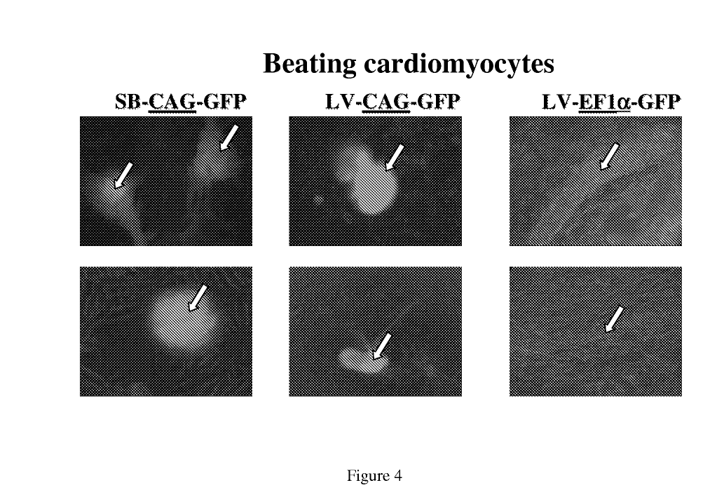

Prior to cell sorting, GFP-expressing undifferentiated HUES cell colonies were manually selected by using a phase contrast light microscope under sterile conditions, to enrich the marker gene expressing population. Such heterogeneous colonies (see FIG. 3A) were harvested from the mouse feeder cells, washed with 1×PBS and sorted based on GFP fluorescence using the FACS Aria High Speed Cell Sorter (Beckton-Dickinson, San Jose, Calif.). Mock-transfected HUES cells were measured to set the level for GFP-positivity with the FACS Diva analysis software; propidium iodide staining was used to gate for the non-viable cells during the sorting procedure. Sorted GFP positive single cells were placed onto the mouse feeder cells and then monitored until the formation of surviving clones. For further gene expression analysis, differentiated cells from GFP-expressing HuES clones were sorted into 4 different artificial fractions based on decreasing GFP fluorescent signal intensity. Cells obtained from different fractions were washed with 1×PBS and immediately resuspended in Trizol® (Invitrogen) for further RNA profile analysis (see Example 7 below).

### Example 6

### Immunohistochemical Assays

For immunostaining, cells were seeded onto eight-well Nunc Lab-Tek II Chambered Coverglass (Nalge Nunc International, Rochester, N.Y.). Cells were fixed with 4% paraformaldehyde in Dulbecco's modified PBS (DPBS) for 15 min at room temperature. Following further washing steps with DPBS, the cells were blocked for 1 hr at room temperature in DPBS containing 2 mg/ml bovine serum albumin, 1% fish gelatin, 0.1% Triton-X 100, and 5% goat serum. The samples were then incubated for 1 hr at room temperature with monoclonal antibodies for stem cell markers Oct-4 (1:50 dilution, Santa Cruz Biotechnology, Santa Cruz, Calif.), SSEA-4 (1:10 dilution, R&D Systems, Minneapolis, Minn.) or podocalyxin (1:10, R&D Systems, Minneapolis, Minn.). Incubations were also carried out using antibodies against the neuron-specific beta III tubulin (1:200 dilution, R&D Systems, Minneapolis, Minn.). After washing with DPBS, the cells were incubated for 1 hr at room temperature with Alexa Fluor 647-conjugated goat anti-mouse IgG antibody. Tric-phalloidin (Sigma-Aldrich, St. Louis, Mo.) staining was used for the detection of actin filaments, in the final concentration of 0.1 μg/ml. DAPI (Invitrogen, Madison, Wis.) was used for nuclear staining, GFP was evaluated by direct fluorescence. The stained samples were studied by an Olympus FV500-IX confocal laser scanning. The blue, green and deep red fluorescence were acquired between 430-460 nm, 505-525 nm, and above 660 nm; using 405 nm, 488 nm, and 633 nm excitations, respectively. All documented measurements were carried out on cells from four independent GFP-expressing clones, at least in triplicate stainings.

### Example 7

### RNA Analysis

RNA isolation was carried out from cells collected in Trizol® reagent (Invitrogen) according to the manufacturer's instruction. cDNA samples were prepared from 0.1 μg total RNA using the Promega Reverse Transcription System Kit as specified by the manufacturer. Tissue specific genes were selected using the TiProD tissue-specific promoter database (see Example 2 above). The following markers were selected: OCT4 and NANOG transcripts as undifferentiated stem cell markers; ACTC, NPPA and PLN genes as cardiac specific markers; PAX6 gene as an early marker for neuronal differentiation; CAPG gene as a skin differentiation marker; PO ribosomal protein and Pol IIA genes as endogenous controls. Pre-developed real-time TaqMan® assays for the listed genes were purchased from Applied Biosystems. For quantifying EFGP mRNA, specific TaqMan® assay were designed for the cDNA sequence. Real-time PCR analyses were carried out using the StepOne™ Real-Time PCR System (Applied Biosystems), according to the manufacturer's instructions.

### Example 8

### Test System

HuES cells are transfected as described in Example 2. differentiated as described in Example 1. Undifferentiated HUES cells with constitutive GFP-expression are selected as described in Example 5 and test and control samples are separated, at least three parallel from each. A test compound assumed to be a modulator of cell differentiation is added to the test sample. HuES cells are differentiated to cardiomyocytes as described in Example 1.

Differentiation of the samples is detected by detecting GFP expression. In a relatively simple variant of the method only fluorescent GFP signal of the cells is detected. If appropriate, morphological signs under phase contrast light microscopy, as well as by immunostaining for various protein markers is observed as described in Example 1 and evaluated vis-a-vis GFP expression. The effect of the assumed modulator drug to cardiomyocyte differentiation is assessed by comparing GFP expression in the test samples and in the control samples.

In a further variant of the method test compounds are added only to the differentiated cardiomyocytes and their effect on GFP activity or cellular conditions are evaluated.

### Example 9

### Cardiomyocyte Test System with Ca-Sensitive Reporter

Concerning cardiomyocyte-focusing test systems, a particularly useful application is to express a Ca-sensitive GFP (GCaMP2) under the control of the “double-feature” CAG promoter. This genetically modified fluorescent protein becomes extremely bright in the presence of Ca2+ [(Tallini et al., PNAS103(12), 4753-8 (2006); Lee et al., PNAS103(35) 13232-7, (2006)] and therefore is a valuable indicator of changes in the internal Ca2+ concentrations due to physiological fluctuations. The normally functioning heart shows regularly periodic Ca2+ waves, the disturbance of which could be a warning sign of pathological processes. In addition, various medically approved drugs could have side effects causing serious arrythmias, based on their partial or complete interference of membrane localized ion channels, such as Ca2+ channels. It is therefore important or may be compulsory to test any proposed potential drugs for their effect on such cellular proteins before the clinical trial.

For such purposes, cardiomyocytes differentiated from transgenic embryonic stem cell clones expressing a CAG promoter-driven GCaMP2 is a particularly useful tool to monitor intracellular Ca2+ fluctuations.

In the present experiment, cardiomyocytes were differentiated as described in Example 1. The basal fluorescent activity of GCaMP2 allowed us to generate undifferentiated transgenic stem cell clones, whereas the strong cardiac-specific activity of the “double-feature” CAG promoter amplifies the periodic signals generated by intracellular Ca2+ waves in the differentiated cardiomyocytes. Drugs known to influence heartbeats (e.g. adrenalin or verapamil) were added to differentiated cell culture. We could show that the effect of drugs known to influence heartbeats can be easily visualized using this fluorescent protein technique, by monitoring the brightness change of GCaMP2 and thereby the frequency of Ca²⁺ waves.

Thus, the test system according to the invention utilizing a Ca-sensitive fluorophore together with the cardiac amplification of the specific signal by the “double-feature” CAG promoter is a valuable fluorescent microscopy-based method and has the potential for replacing patch clamp-based procedures for high-throughput drug screenings.

### INDUSTRIAL APPLICABILITY

The present invention is useful in providing test systems based on stem cells or stem cell differentiation. The double-feature promoter of the invention allows detection of successful gene delivery into stem cells by its constitutive feature. Upon differentiation of the cells expression driven by the double-feature promoter of the invention is increased thereby the differentiation status of the cells can be monitored. Moreover expression of genes of interest, e.g. reporter genes or genes the product of which presumably affects differentiation can be driven by the double-feature promoter of the invention.

By differentiating the stem cells e.g. to cardiomyocytes a usefuly test system for drugs presumably effecting heart muscle state or tissue differentiation is prepared.

The system described herein is particularly useful in drug screening.

1. A stem cell or a cell differentiated therefrom containing a copy of a stably inserted or inheritable expression construct that is suitable for the expression of transgenes in stem cells, said construct comprising:
at least a double-feature promoter being operable both in stem cells and in differentiated tissues, wherein said promoter is
constitutive while
the expression level thereof being subject to a tissue or cell type specific regulation in differentiated cells,
and, optionally,
under the control of said promoter, a transgene, wherein said transgene is expressed in said stem cell.

2. The stem cell according to claim 1, wherein said promoter is selected from a group consisting of a fusion promoter, a double-feature CAG promoter; a cardiac actin promoter and a human albumin promoter.

3. The stem cell according to claim 2, wherein said double-feature CAG promoter contains a Cytomegalovirus (CMV) Immediate Early Enhancer, two parts of the chicken beta-actin promoter and part of the rabbit beta1-globin promoter, preferably the nucleotide sequence of said CAG promoter is the sequence given in SEQ ID NO:1 or its functional double-feature homologue.

4. The stem cell according to claim 1, wherein said expression construct further comprises sequences selected from the following group:
viral transduction sequences,
transposon derived sequences,
a pair of sequences allowing site-specific recombination upstream of the promoter and downstream of said transgene.

5. The stem cell according to claim 1, wherein said transgene is selected from
a reporter gene and a reporter protein encoded by said reporter gene is expressed in said stem cell,
a construct for the expression of a siRNA and a connected reporter gene, and said siRNA and said reporter protein encoded by said reporter gene are expressed in said stem cell or in its differentiated derivative,
a gene encoding for a fluorescent protein preferably selected from a group comprising Green Fluorescent Protein (GFP), Ca-sensitive GFP (GCaMP2), Red Fluorescent Protein (RFP), Yellow Fluorescent Protein (YFP), Cyan Fluorescent Protein (CFP), DS-redMST Fluorescent Protein; Luciferase; β-galactosidase; a sensor fluorescence protein for reporting cellular ion concentrations or membrane potential and
a gene encoding for a protein providing resistance to chemical selecting agents, preferably to puromycin, neomycin or other cytotoxic agents,
wherein
preferably said reporter protein gives rise to a detectable signal the intensity of which correlates with the activity of said promoter or said reporter protein gives specific resistance to a chemical agent which resistance correlates with cellular conditions or the activity of said promoter.

6. The stem cell defined or used in claim 1, wherein said stem cell
is produced without causing any harm to a human embryo,
is not derived from any cell that was produced by causing any harm to a human embryo and/or
is not of human embryonic origin.

7. The stem cell according to claim 1, wherein said stem cell is a human embryonic stem cell (HUES) or a human derived induced pluripotent stem (iPS) cell.

8. A cell differentiated from the stem cell according to claim 1, wherein said stem cell comprises a stably inherited or inserted expression construct that is suitable for expression of a transgene in stem cells, said construct comprising
at least a double-feature promoter being operable both in stem cells and in differentiated tissues, wherein said promoter is
constitutive while
the expression level thereof being subject to a tissue or cell type specific regulation in differentiated cells,
and, optionally,
a transgene under the control of said promoter.

9. The differentiated cell according to claim 8 wherein said promoter is a double-feature CAG promoter, wherein said CAG promoter preferably contains a Cytomegalovirus (CMV) Immediate Early Enhancer, two parts of the chicken beta-actin promoter and part of the rabbit beta1-globin promoter, preferably the nucleotide sequence of said CAG promoter is the sequence given in SEQ ID NO:1 or its functional double-feature homologue.

10. The differentiated cell according to claim 8, wherein said cell is a cardiomyocyte.

11. The stem cell according to claim 8, wherein said expression construct further comprises sequences selected from the following group:
viral transduction sequences,
transposon derived sequences,
a pair of sequences allowing site-specific recombination upstream of the promoter and downstream of said transgene.

12. The stem cell according to claim 8, wherein said transgene is selected from
a reporter gene wherein the reporter protein encoded by said reporter gene is expressed in said stem cell,
a construct for the expression of a siRNA and a connected reporter gene, and said siRNA and said reporter protein encoded by said reporter gene are expressed in said stem cell or in its differentiated derivative,
a gene encoding for a fluorescent protein preferably selected from a group comprising Green Fluorescent Protein (GFP), Ca-sensitive GFP (GCaMP2), Red Fluorescent Protein (RFP), Yellow Fluorescent Protein (YFP), Cyan Fluorescent Protein (CFP), DS-redMST Fluorescent Protein; Luciferase; β-galactosidase; a sensor fluorescence protein for reporting cellular ion concentrations or membrane potential and
a gene encoding for a protein providing resistance to chemical selecting agents, preferably to puromycin, neomycin or other cytotoxic agents,
wherein
preferably said reporter protein gives rise to a detectable signal the intensity of which correlates with the activity of said promoter or said reporter protein gives specific resistance to a chemical agent which resistance correlates with cellular conditions or the activity of said promoter.

13. Method for producing a stem cell or a differentiated stem cell,
said stem cell or differentiated stem cell comprising
a stably inserted or inheritable expression construct that is suitable for expression of a transgene in stem cells, said construct comprising
at least a double-feature promoter being operable both in stem cells and in differentiated tissues, wherein said promoter is
constitutive while
the expression level thereof being subject to a tissue or cell type specific regulation in differentiated cells,
and, optionally,
a transgene under the control of said promoter;
said method comprising
i) providing a construct that comprises—at least a constitutive double-feature promoter being operable both in stem cells and in differentiated tissues, and having an expression level being subject to a tissue or cell type specific regulation in differentiated cells, and, optionally,
under the control of said promoter, a transgene, wherein said transgene is expressed in said stem cell,
ii) introducing the construct defined in step i) into a stem cell and, optionally,
iii) differentiating said stem cell.

14. The method of claim 13, wherein said introduction of said construct in step ii) is done by viral transduction or by using a transposon-transposase system.

15. The method according to claim 13, wherein said promoter is selected from a group consisting of a fusion promoter, a double-feature CAG promoter; cardiac actin promoter; and human albumin promoter, and
wherein preferably said double-feature CAG promoter contains the Cytomegalovirus (CMV) Immediate Early Enhancer, two parts of the chicken beta-actin promoter and part of the rabbit beta1-globin promoter, preferably the nucleotide sequence of said CAG promoter is the sequence given in SEQ ID NO:1 or its functional double-feature homologue.

16. The method according to claim 13, wherein said transgene is selected from
a reporter gene wherein the reporter protein encoded by said reporter gene is expressed in said stem cell,
a construct for the expression of a siRNA and a connected reporter gene, and said siRNA and said reporter protein encoded by said reporter gene are expressed in said stem cell or in its differentiated derivative,
a gene encoding for a fluorescent protein preferably selected from a group comprising Green Fluorescent Protein (GFP), Ca-sensitive GFP (GCaMP2), Red Fluorescent Protein (RFP), Yellow Fluorescent Protein (YFP), Cyan Fluorescent Protein (CFP), DS-redMST Fluorescent Protein; Luciferase; β-galactosidase; a sensor fluorescence protein for reporting cellular ion concentrations or membrane potential and
a gene encoding for a protein providing resistance to chemical selecting agents, preferably to puromycin, neomycin or other cytotoxic agents,
wherein
preferably said reporter protein gives rise to a detectable signal the intensity of which correlates with the activity of said promoter or said reporter protein gives specific resistance to a chemical agent which resistance correlates with cellular conditions or the activity of said promoter.

17. A method for testing the effect of a test compound on a stem cell or a differentiated stem cell as defined in claim 1,
said method comprising
contacting said stem cell or a differentiated stem cell with a test compound,
determining the effect of said test compound on the amount of said reporter gene product or activity in the differentiated cells as compared to a control sample.

18. The method according to claim 17 for identifying or profiling a modulator of cell differentiation comprising:
i) contacting a test sample comprising a stem cell according to claim 1 with a test compound, selected from a group including drugs, hormones, artificial or natural compounds, and
ii) allowing said stem cell to differentiate;
iii) determining the effect of said test compound on the amount of said reporter gene product or activity in the differentiated cells as compared to a control sample,
or
a) providing a stem cell according to claim 1
b) allowing said stem cell to differentiate;
c) contacting the stem cells or their differentiated derivatives with a test compound and
d) measuring the changes in the reporter fluorescent agent thereby indicating cellular conditions.

19. A method according to claim 17 for monitoring stem cell differentiation comprising:
i) providing a stem cell according to claim 1,
ii) monitoring the expression level of said reporter gene or the amount or activity of said reporter gene product, and
iii) drawing conclusions regarding the direction of the differentiation of said stem cell based on the expression level of said reporter gene in any of the cells differentiated from said stem cell, or
iv) drawing conclusions wherein said monitoring is done by measuring the intensity of the signal emitted by the product of said reporter gene in the stem cells or their differentiated derivatives.

20. A reagent kit comprising:
a stem cell defined or used in any preceding claim, and
one or more test reagents.
